(19) United States
(12) Patent Application Publication
(10) Pub. No.: US 2020/0388404 A1
(43) Pub. Date: Dec. 10, 2020

(54) SYSTEM FACILITATING HEALTHCARE SERVICES AND A METHOD THEREOF

(71) Applicant: Ami Anand SHAH, (US)

(72) Inventors: Ami Anand SHAH, New York, NY (US); Raj JHAVERI, Orange, CA (US)

(21) Appl. No.: 16/893,139

(22) Filed: Jun. 4, 2020

Related U.S. Application Data
(60) Provisional application No. 62/857,178, filed on Jun. 4, 2019.

(51) Int. Cl. G16H 80/00; G16H 40/20; G06Q 20/28; G06Q 50/20; G16H 10/60; G16H 50/70; G16H 15/00; G06Q 50/26; G06F 9/451; G16H 40/67

(52) U.S. Cl. CPC G16H 80/00; G16H 40/20; G06Q 20/28; G06Q 50/2057; G16H 40/67; G16H 50/70; G16H 15/00; G06Q 50/265; G06F 9/451; G16H 10/60

(57) ABSTRACT

The present disclosure relates to system and method facilitating healthcare services.

Plurality of designated portals are designed for providing the healthcare services. Request including learning request or consult request for availing a healthcare service through one or more providers is received by a user. Learning request and the consult request are directed to a designated portal selected by the user. Designated portal controls display of the details of the user to the one or more providers, according to the learning request or the consult request. Request results are generated for the learning request and the consult request, through the designated portal. The request result is generated in a preselected mode of access and according to a user's preference, such that the user avails the healthcare services by accessing the request result.

### CROSS-REFERENCE TO RELATED APPLICATIONS AND PRIORITY

The present application claims priority from U.S. Provisional Patent Application No. 62/857,178 filed on 4 Jun. 2019, the entity of which is hereby incorporated by reference.

### TECHNICAL FIELD

The present disclosure in general relates to a field of service management and facilitation. More particularly, the present disclosure relates to the system and method facilitating healthcare services.

### BACKGROUND

Patients, particularly minors and people with challenges, may be afflicted with ailments difficult to describe. Such patients may not be as verbally articulate as normally healthy adult patients. Symptoms may change over time for a particular affliction or group of afflictions. Such patients further may not have the necessary capabilities or apparatus to search for information online.

Symptoms of mental disorders may be different or less obvious in minors and disabled or challenged persons. Memory difficulties or personality changes may be difficult to describe by the patient or identify by professionals. Even physical aches or pain, fatigue, loss of appetite, sleep problems or loss of interest in some activities may also be difficult to clearly identify. Unlike signs picked up on physical exams or laboratory results, symptoms are what the patient experiences first-hand. While normally healthy adults can communicate symptoms clearly, children and others may be unable to provide professionals with essential information.

Furthermore, many a times in urgent situations, direct physical visit to a medical practitioner may not be possible due to certain challenging reasons, such as, lack of information about experts in field, geo-location issues, late understanding on symptoms and alike. Furthermore, in most of the scenarios, patients find it difficult to understand their first aid medical requirement due to improper knowledge and also hesitate to avail direct or follow-up appointments due to larger waiting time.

Patients also find it difficult to connect with correct knowledge imparting sources where a healthy discussion or understanding may be provided before actually directly investing into a medical consultation or follow-ups.

### SUMMARY

Before the present system and method facilitating healthcare services in a storage system is described, it is to be understood that this application is not limited to the particular systems, and methodologies described, as there can be multiple possible embodiments which are not expressly illustrated in the present disclosure. It is also to be understood that the terminology used in the description is for the purpose of describing the particular versions or embodiments only, and is not intended to limit the scope of the present application. This summary is provided to introduce concepts related to systems and methods facilitating healthcare services in a communication network. This summary is not intended to identify essential features of the claimed subject matter nor is it intended for use in determining or limiting the scope of the claimed subject matter.

In one implementation, a system facilitating healthcare services is described. The system comprises of a User Interface (UI), configured with a plurality of designated portals, designed for providing the healthcare services, a memory and a processor coupled to the memory. The memory stores a plurality of instructions to be executed by the processor. The processor is configured to receive, from a user, through the UI, a request for availing a healthcare service through one or more providers. The request comprises at least one of a learning request or a consult request. The processor is further configured to direct, the at least one of the learning request and the consult request to a designated portal of the plurality of designated portals selected by the user. The designated portal controls display of the details of the user to the one or more providers, according to the at least one of the learning request and the consult request, the processor is further configured to generate, a request result for at least one of the learning request and the consult request, through the designated portal. The request result is generated in a preselected mode of access and according to a user's preference, such that the user avails the healthcare services by accessing the request result.

In another implementation, an electronic device facilitating healthcare services is described. The electronic device comprises of a User Interface (UI), a memory and a processor coupled to the memory. The memory stores a plurality of instructions to be executed by the processor. The processor is configured to register, through the UI, details of the user for submitting a request for availing a healthcare service through one or more providers. The request comprises at least one of a learning request or a medical assistance request. The processor is further configured to select, a designated portal of the plurality of portals according to at least one of the learning request and the medical assistance request. The designated portal controls display of the details of the user to the one or more providers, according to the at least one of the learning request and the medical assistance request. The processor is further configured to receive, a request result for at least one of the learning request and the medical assistance request, through the designated portal, wherein the request result is accessed by a user in a preselected mode of access, wherein the request result is received according to a user's preference.

In another implementation, an electronic device facilitating healthcare services is described. The electronic device comprises of a User Interface (UI) with a dashboard showing a plurality of designated portals, a memory and a processor coupled to the memory. The memory stores a plurality of instructions to be executed by the processor. The processor is configured to receive, through the UI, a request for availing a healthcare service from one or more users. The request comprises at least one of a learning request or a medical assistance request, wherein the healthcare services are availed by connecting with one or more providers. The processor is further configured to select, a designated portal of the plurality of portals according to at least one of the learning request and the medical assistance request. The designated portal controls display of the details of the user to the one or more providers, according to the at least one of the learning request and the medical assistance request. The processor is further configured to generate, a request result for at least one of the learning request and the medical assistance request, through the designated portal. The request result is generated in a preselected mode of access, wherein the request result is generated according to a user's preference. such that the user accesses the request result for availing the healthcare services.

In one implementation, a method facilitating healthcare services is described. The method comprises of receiving, from a user, through the UI, a request for availing a healthcare service through one or more providers. The request comprises at least one of a learning request or a consult request. The method further comprises directing, the at least one of the learning request and the consult request to a designated portal of a plurality of designated portals selected by the user. The designated portal controls display of the details of the user to the one or more providers, according to the at least one of the learning request and the medical assistance request. The method further comprises generating, a request result for at least one of the learning request and the medical assistance request, through the designated portal. The request result is generated in a preselected mode of access and the request result is generated according to a user's preference. such that the user avails the healthcare services by accessing the request result.

In another implementation, a method facilitating healthcare services is described. The method comprises registering, through the UI, details of the user for submitting a request for availing a healthcare service through one or more providers. The request comprises at least one of a learning request or a consult request. The method further comprises selecting, a designated portal of the plurality of portals according to at least one of the learning request and the consult request. The designated portal controls display of the details of the user to the one or more providers, according to the at least one of the learning request and the consult request. The method further comprises receiving, a request result for at least one of the learning request and the consult request, through the designated portal, wherein the request result is accessed by a user in a preselected mode of access, wherein the request result is received according to a user's preference.

In another implementation, a method facilitating healthcare services is described. The method comprises receiving, through the UI, a request for availing a healthcare service from one or more users. The request comprises at least one of a learning request or a consult request. The healthcare services are facilitated through the one or more providers. The method further comprises selecting, a designated portal of the plurality of portals according to at least one of the learning request and the consult request. The designated portal controls display of the details of the user to the one or more providers, according to the at least one of the learning request and the consult request. The method further comprises generating, a request result for at least one of the learning request and the consult request, through the designated portal. The request result is generated in a preselected mode of access and the request result is generated according to a user's preference, such that the user accesses the request result for availing the healthcare services.

### BRIEF DESCRIPTION OF DRAWINGS

The detailed description is described with reference to the accompanying figures. In the figures, the left-most digit(s) of a reference number identifies the figure in which the reference number first appears. The same numbers are used throughout the drawings to refer like features and components.

FIG. 1 illustrates a network implementation of a system 102 facilitating healthcare services, in accordance with an embodiment of the present subject matter.

FIG. 2 illustrates module details of the system 102 facilitating healthcare services, in accordance with an embodiment of the present subject matter.

FIG. 3 illustrates an electronic device for a user or a provider facilitating healthcare services, in accordance with an embodiment of the present subject matter.

FIG. 4 illustrates a flow chart for method 400 facilitating healthcare services through the system 102, in accordance with an embodiment of the present subject matter.

FIG. 5(a) illustrates a flowchart for a method 500 facilitating healthcare services to a user through the electronic device 300, in accordance with an embodiment of the present subject matter.

FIG. 5(b) illustrates a flowchart for a method 500 facilitating healthcare services through a provider through the electronic device 300, in accordance with an embodiment of the present subject matter.

FIG. 6 illustrates an exemplary dashboard for the user, configured over a user interface of the electronic device 300 facilitating healthcare services, in accordance with an embodiment of the present subject matter.

FIG. 7 illustrates an exemplary dashboard for the provider(s), configured over the user interface of the electronic device 300 facilitating healthcare services, in accordance with an embodiment of the present subject matter.

FIG. 8(a) illustrates an exemplary view of the UI of the electronic device 300 facilitating healthcare services to the user, in accordance with an embodiment of the present subject matter.

FIG. 8(b) illustrates another exemplary view of the UI of the electronic device 300 facilitating healthcare services to the user, in accordance with an embodiment of the present subject matter.

FIG. 9(a) illustrates an exemplary view of the UI of the electronic device 300 facilitating healthcare services through the providers, in accordance with an embodiment of the present subject matter.

FIG. 9(b) illustrates another exemplary view of the UI of the electronic device 300 facilitating healthcare services through the providers, in accordance with an embodiment of the present subject matter.

FIG. 10 illustrates an exemplary view of the UI of the system 102 facilitating healthcare services to the users through the providers, in accordance with an embodiment of the present subject matter.

### DETAILED DESCRIPTION

Some embodiments of the present disclosure, illustrating all its features, will now be discussed in detail. The words “comprising”, “receiving”, “determining”, “assigning” and other forms thereof, are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a”, “an” and “the” include plural references unless the context clearly dictates otherwise. Although any systems and methods similar or equivalent to those described herein can be used in the practice or testing of embodiments of the present disclosure, the exemplary, systems and methods for assigning dynamic operation of devices in a communication network are now described. The disclosed embodiments of the systems and methods for assigning dynamic operation of devices in a communication network are merely exemplary of the disclosure, which may be embodied in various forms.

Various modifications to the embodiment will be readily apparent to those skilled in the art and the generic principles herein may be applied to other embodiments. However, one of ordinary skill in the art will readily recognize that the present disclosure for assigning dynamic operation of devices in a communication network is not intended to be limited to the embodiments illustrated, but is to be accorded the widest scope consistent with the principles and features described herein.

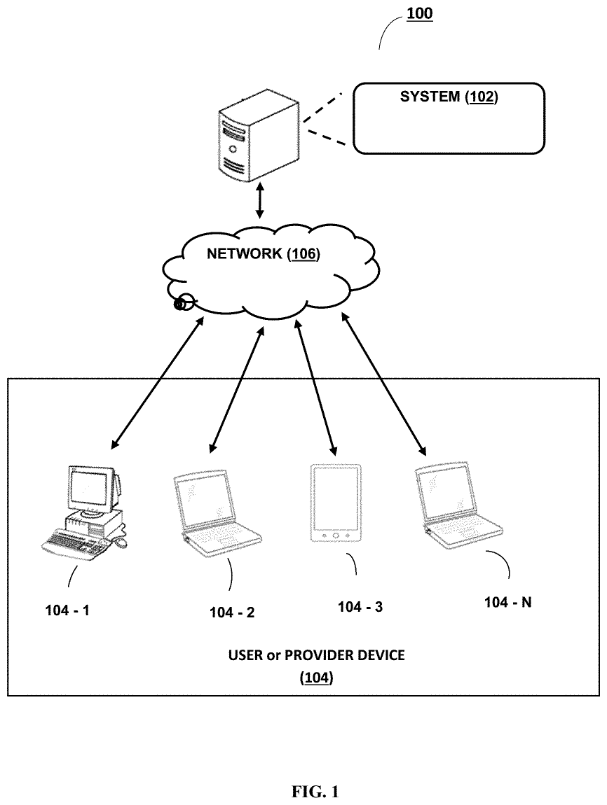

Referring now to FIG. 1, a network implementation 100 of a system 102 facilitating healthcare services is disclosed. Although the present subject matter is explained considering that the system 102 is implemented on a server, it may be understood that the system 102 may also be implemented in a variety of computing systems, such as a laptop computer, a desktop computer, a notebook, a workstation, a mainframe computer, a server, a network server, and the like. In one implementation, the system 102 may be implemented over a cloud network. Further, it will be understood that the system 102 may be accessed by multiple users or multiple providers through one or more user devices 104-1, 104-2 . . . 104-N, collectively referred to as user device 104 (or provider device 104) hereinafter, or applications residing on the user device 104. Examples of the user device 104 may include, but are not limited to, a portable computer, a personal digital assistant, a handheld device, and a workstation. The user device 104 may be communicatively coupled to the storage system 102 through a network 106. The user device may also be referred as an electronic device (explained in later sections).

In one implementation, the network 106 may be a wireless network, a wired network or a combination thereof. The network 106 may be implemented as one of the different types of networks, such as intranet, local area network (LAN), wide area network (WAN), the internet, and the like. The network 106 may either be a dedicated network or a shared network. The shared network represents an association of the different types of networks that use a variety of protocols, for example, Hypertext Transfer Protocol (HTTP), Transmission Control Protocol/Internet Protocol (TCP/IP), Wireless Application Protocol (WAP), and the like, to communicate with one another. Further, the network 106 may include a variety of network devices, including routers, bridges, servers, computing devices, storage devices, and the like.

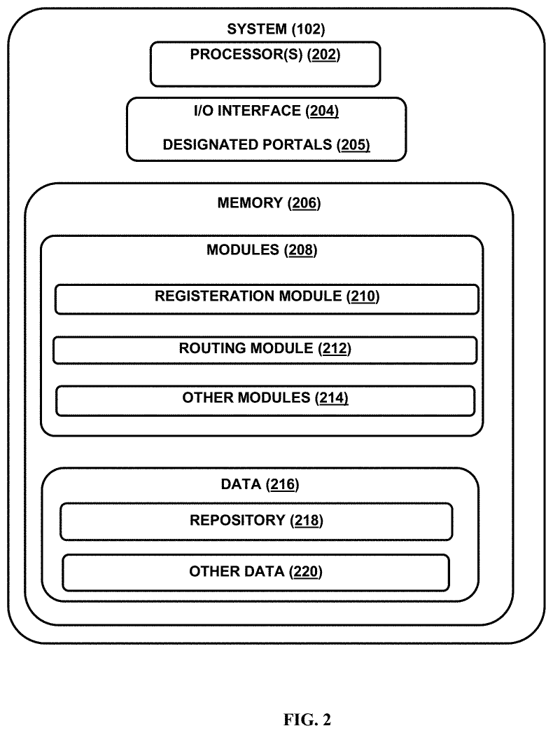

Referring to FIG. 2, configuration of the system 102 is shown. In one embodiment, the storage system 102 may include at least one processor 202, an input/output (I/O) interface 204, and a memory 206. The at least one processor 202 may be implemented as one or more microprocessors, microcomputers, microcontrollers, digital signal processors, central processing units, state machines, logic circuitries, and/or any devices that manipulate signals based on operational instructions. Among other capabilities, at least one processor may be configured to fetch and execute computer-readable instructions stored in the memory 206.

The I/O interface 204 may include a variety of software and hardware interfaces, for example, a web interface, a graphical user interface, and the like. The I/O interface 204 may allow the system 102 to interact with the user directly or through the user device 104. Further, the I/O interface 204 may enable the system 102 to communicate with other computing devices, such as web servers and external data servers (not shown). The I/O interface 204 may facilitate multiple communications within a wide variety of networks and protocol types, including wired networks, for example, LAN, cable, etc., and wireless networks, such as WLAN, cellular, or satellite. The I/O interface may include one or more ports for connecting a number of devices to one another or to another server.

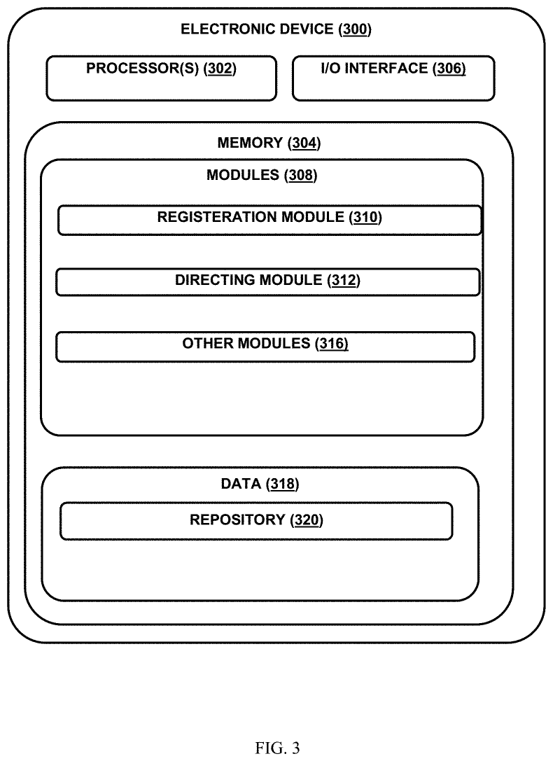

In one implementation, a user or the provider may access the storage system 102 via an I/O interface of the electronic device 300 (details explained in FIG. 3). The user or provider may be registered using the I/O interface in order to use the system 102. In one aspect, the user may access the I/O interface of the system 102 for obtaining information, or the provider may access the system 102 for providing the information or configuring the system 102.

The memory 206 may include any computer-readable medium known in the art including, for example, volatile memory, such as static random access memory (SRAM) and dynamic random access memory (DRAM), and/or non-volatile memory, such as read only memory (ROM), erasable programmable ROM, flash memories, hard disks, optical disks, and magnetic tapes.

The memory 206 is connected to a plurality of modules 208. The modules 208 may be configured within the memory 208 as software modules 208 or the modules 208 may be connected to each of the processor 202, the I/O interface 204 and the memory 206 as hardware modules. The modules 208 comprises a registration module 210, a routing module 212, and other modules 214. The system 102 also comprise data 216 stored in a repository 218 and other data 220. The data 216 comprises details of each of the user (patient or guardian) and the providers.

In an exemplary embodiment, referring to FIG. 1 and FIG. 2 in combination, the system 102 facilitating healthcare services is shown. The system 102 comprises of the User Interface (UI) 106 configured with a plurality of designated portals 205. Each portal designed for providing the healthcare services according to a user's requirement. Each of the designated portal 205 is displayed over the UI 106 of the system 102 as well as electronic device of the user and the provider (explained later).

The system 102 further comprises the memory 104 and the processor 102 coupled to the memory 104. The memory 106 stores a plurality of instructions (modules) to be executed by the processor 102. The processor 102 is configured to receive a request for availing a healthcare service through one or more users. The request may be received from the user through the electronic device 300 (user device 104). The user comprises a patient, a guardian of a minor patient, a family member of the patient, wherein the user is a registered user provided with user credentials for accessing the system 102.

In accordance with an embodiment, referring to FIG. 3, the user accesses the system 102 through the electronic device 300. The electronic device 300 comprises a processor 302, a memory 304 coupled to the processor 302, and a user interface 306. The memory 304 stores a plurality of instructions (modules 308) to be executed by the processor 302. The processor 302 is configured to enable registration of the user through the UI 306 by using the registration module 310. The directing module 312 further directs the user to the designated portals 205. The electronic device 300 further comprises other modules 316 for other operations executed by the processor 302. The electronic device further comprises data 318 stored in a repository 320. The data 318 comprises details of the user.

When the user accesses the system 102, the user is provided with a form to be filled in order to submit the request for availing the healthcare service. The user then submits details in the form for submitting a request for availing the healthcare service through one or more providers. Once the details are filled by the user towards the registration, the user is provided with login credentials for accessing the system 102. The user's profile created by the system 102 may be stored in the repository 320 of the electronic device 300 and also with the repository 218 of the system 102.

The request comprises at least one of a learning request and the consult request. The details filled by the user are protected and the display and access of the details is controlled through the designated portal 205 coupled to the processor 202. Details relevant for the request are only displayed, while remaining details remains locked as per locking rules preconfigured within the system 102 or display preference selected and enabled by the user.

The details comprise at least one of personal details of the user, a medical history of the user, medical reports or records of the user, insurance details of the user, contact details of the user. The personal details comprise name, contact details, date of birth, gender, family details etc. In case of a minor patient, details of guardian are also stored in the system 102.

The request comprises at least one of a learning request or a consult request (may also be referred as a medical assistance request). The request comprises a prepaid package request for availing the healthcare service. The prepaid package comprises at least one of the learning request or a consult request. The prepaid request may be availed by user by making a payment through a payment designated portal of the system and then the user may subscribe for the learning request and the consult request as defined in the package.

In an example embodiment, the consult request comprises at least one of a preliminary medical guidance request, an appointment request for expert medical consultation with one or more providers, reschedule of one of the learning request or the medical assistance request, a cancellation of one of the learning request or the medical assistance request, a follow-up appointment request for expert medical consultation with one or more providers. The learning request comprises a request for accessing a knowledge resource configured within the system 102, e-learning request, a webinar, a workshop, a community connection and alike.

In an exemplary embodiment, referring to FIG. 3, the electronic device 300 is also used by the one or more providers for accessing the system 102. The provider accesses the system 102, only after the registration similar to the user. The provider also fills a form with provider's personal details. Once the provider's personal details are filled, the login credentials are generated for the provider for accessing the system 102. The provider's profile is created by the system 102 and may be stored in the repository 320 of the electronic device 300 and also with the repository 218 of the system 102.

Once the request is received, the request is directed to one of the designated portal 205 of the plurality of designated portals. The request is then viewed by the provider through the UI 306 of the electronic device 300. The user and the provider may also select the designated portal 205 from the plurality of portals according to at least one of the learning request and the consult request. The designated portal controls display of the details of the user to the one or more providers, according to the at least one of the learning request and the consult request.

The designated portal 205 comprises an on-demand medical guidance portal, a medical expert consultation portal, a health monitoring portal, an e-learning portal, a message portal, a calendar portal, a payment portal, a user profile portal generated after registration of the user for displaying details of the user according to the request, a help portal, a feedback portal, a review portal.

The designated portal 205 controls display of the details of the user to the one or more providers, according to selection of the at least one of the learning request and the consult request. The one or more providers comprises at least one of medical experts, non-medical experts, doctors, medical counsellors, non-medical counsellors, psychologists, trainers. The one or more providers are registered providers accessing the system 102, through the electronic device 300, by using login credentials of the providers generated by the system 102.

The processor 202 then generates, a request result for at least one of the learning request and the consult request, through the designated portal 205. The request result is generated in a preselected mode of access and according to a user's preference, such that the user may further avail the healthcare services by accessing the request result through the UI 306. The user's preference comprises an age preference of the provider, a gender preference of the provider, a community preference of the provider, a language preference of the provider, a location preference of the provider and alike.

The request result comprises at least one of a preliminary consultation for an on-demand medical guidance received as the consult request, a medical expert consultation towards a medical issue, with one or more providers for a medical appointment request received as the consult request, a medical report for a health monitoring request received as the consult request, a list of courses, webinars, workshops, community learning, generated for the request received as the learning request. The user's preference also comprises a preference in selection of a provider from a list of the providers for availing the healthcare services.

The request result is received by the user for at least one of the learning request and the consult request, through the designated portal 205. The request result is accessed by the user in the preselected mode of access. The request result is received according to the user's preference. The preselected mode of access comprises one of an audio mode, a video mode, a text mode, an email mode. The request result comprises one of a real-time request result or a time-slot defined request result.

In an exemplary embodiment, the system 102 is also configured prioritize the request for a follow-up appointment received as the consult request according to the details of the user. The details comprise medical history of a patient, frequency of appointment of the patient, health monitoring results as generated by the system. The request is then directed to the designated portal 205 for scheduling the follow-up appointment according to the prioritizing. The prioritizing sets an order number of the follow-up appointment in a list of appointments.

In another exemplary embodiment, the processor 202 is also configured to identify correlation patterns according to the details of the user and previously generated request results for the user. The correlation pattern is identified through a monitoring portal of the plurality of designated portals 205. The designated portal then recommends a subsequent request result according to the correlation pattern. The subsequent request result comprises a follow-up appointment with one or more medical expert or the non-medical expert, or learning schedules.

The system 102 further comprises a communication module (not shown) coupled to the processor 202. The communication module is configured for establishing a communication between the electronic device 300 and the system 102. The communication module also enables a communication between the user and the one or more providers assisting in availing of the request result.

The communication module is configured to trigger each of an alarm towards a medical emergency or a non-medical emergency, and reminders for availing the healthcare service through request results. Such alarms and reminders are displayed over the UI 306 of the user and the provider(s).

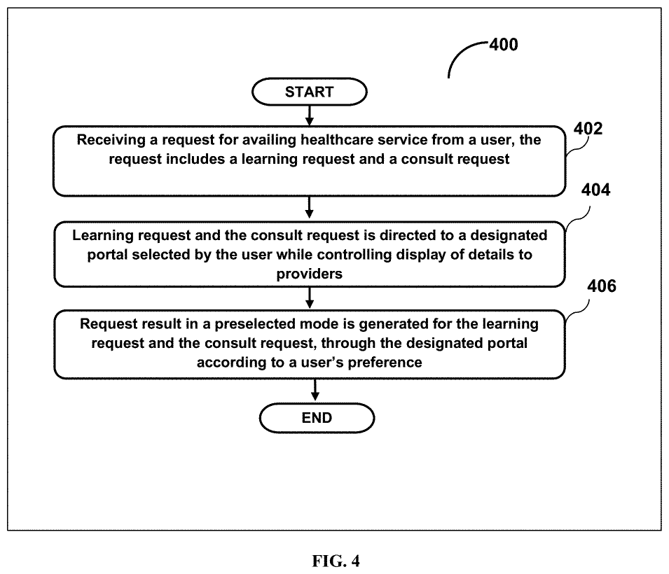

Referring now to FIG. 4, a method 400 facilitating healthcare services, is disclosed in accordance with an embodiment of the present subject matter. The method 400 may be described in the general context of computer executable instructions. Generally, computer executable instructions can include routines, programs, objects, components, data structures, procedures, modules, functions, and the like, that perform particular functions or implement particular abstract data types. The method 400 and the method 500 may also be practiced in a distributed computing environment where functions are performed by remote processing devices that are linked through a communications network. In a distributed computing environment, computer executable instructions may be located in both local and remote computer storage media, including memory storage devices.

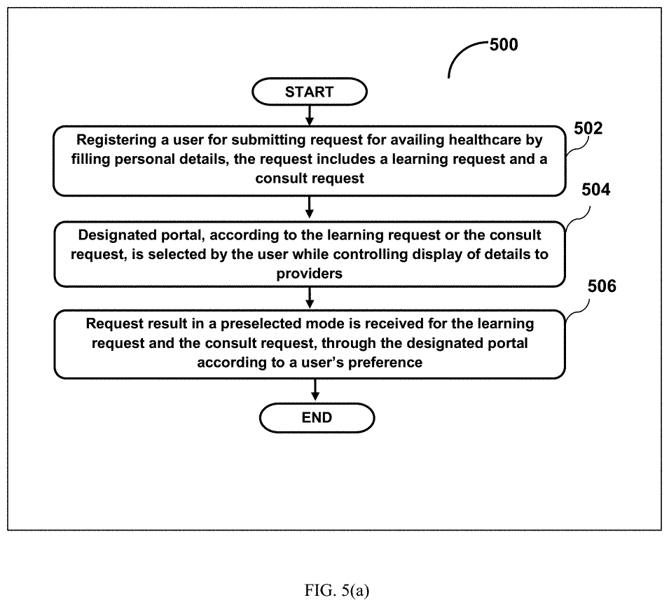

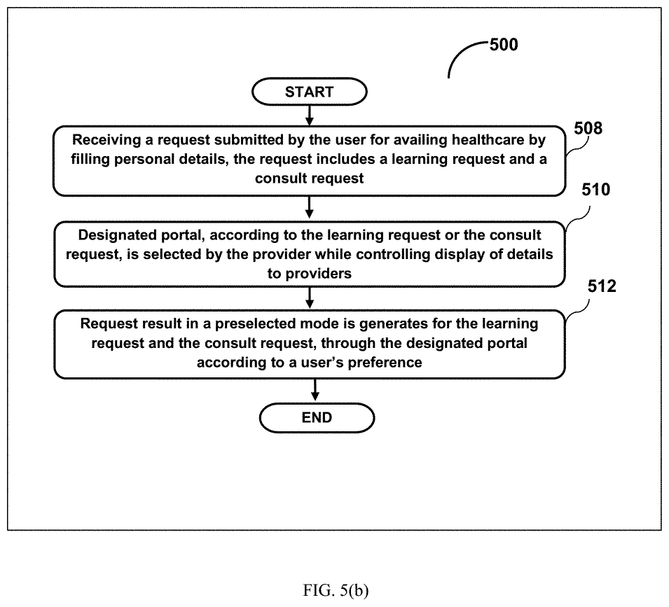

The order in which the method 400 and the method 500 is described is not intended to be construed as a limitation, and any number of the described method blocks can be combined in any order to implement the method 400 the method 500 or alternate methods. Additionally, individual blocks may be deleted from the method 400 and the method 500 without departing from the spirit and scope of the subject matter described herein. Furthermore, the method 400 the method 500 can be implemented in any suitable hardware, software, firmware, or combination thereof. However, for ease of explanation, in the embodiments described below, the method 400 in FIG. 4 may be considered to be implemented in the above described storage system 102, the method 500 in FIG. 5(a) and FIG. 5(b) may be implemented by the electronic device 300.

At block 402, the request for availing the healthcare service through one or more providers is from by the user. The request comprises at least one of the learning request and the consult request.

At block 404, the at least one of the learning request and the consult request is directed to the designated portal 205 of the plurality of designated portals selected by the user. The designated portal 205 controls display of the details of the user to the one or more providers, according to the at least one of the learning request and the consult request.

At block 406, the request result is generated for at least one of the learning request and the consult request, through the designated portal 205. The request result is generated in a preselected mode of access and according to a user's preference, such that the user avails the healthcare services by accessing the request result.

In another exemplary embodiment, referring to FIG. 5(b), the method 500 facilitating the healthcare services is shown. The method 500 illustrates steps user's side flow chart submitting request for accessing the healthcare service by connecting with the system 102 and the one or providers.

At step 502, the user is registered by submitting details for submitting the request for availing a healthcare service through one or more providers. The request comprises at least one of the learning request or the consult request.

At step 504, the designated portal of the plurality of portals is selected according to at least one of the learning request and the consult request. The designated portal controls display of the details of the user to the one or more providers, according to the at least one of the learning request and the consult request.

At step 506, the request result is received for at least one of the learning request and the consult request, through the designated portal. The request result is accessed by a user in a preselected mode of access. The request result is generated according to the user's preference.

In another exemplary embodiment, referring to FIG. 5(b), the method 500 facilitating the healthcare services is shown. The method 500 illustrates the provider's side flow chart accessing the request to further facilitate the healthcare service by connecting with the system 102 and the users.

At step 508, the provider receives the request for availing a healthcare service from one or more users. The request comprises at least one of the learning request and the consult request. The healthcare services are facilitated through the one or more providers.

At step 510, the designated portal of the plurality of portals is selected according to at least one of the learning request and the consult request. The designated portal controls display of the details of the user to the one or more providers, according to the at least one of the learning request and the consult request.

At step 512, the request result is generated for at least one of the learning request and the consult request, through the designated portal. The request result is accessed by a user in a preselected mode of access. The request result is generated according to the user's preference.

In an exemplary embodiment, detailed working of the system 102 executing the method 400 and the electronic device(s) executing the method 500 will now be explained.

The patient may submit the request for the preliminary consultation as the on-demand preliminary consultation. The system 102 searches through the database 218 and generates one of a list of medical experts or non-medical experts (providers) hired for providing the on-demand preliminary consultation, or the system 102 may auto-generate the request result. In case of the list, the user may select one or more medical experts or non-medical experts simultaneously. The on-demand preliminary consultation is an advisory consultation.

In another exemplary scenario, the patient may submit the request for the consultation (a medical consultation). The system 102 searches through the database 218 and generates one of the list of the medical experts and profiles (providers) hired for providing the medical consultation, or the system 102 may also auto-generate the request result. In case of the list, the user may select one or more medical experts for a simultaneous consultation. The medical consultation is availed in one an audio mode through telephone, audio-video mode through any of the app like skype, WhatsApp and alike.

In another exemplary scenario, the patient may submit the learning request for accessing a learning content or for connecting with other user's with similar requirements. The system 102 then generates a list of learning contents (through designated portal 205) where the user may select the content according to user's choice. The system 102 also routes the user to the designated portal 205 showing a list of other user's (community connection) to be connected by the user. The other user's may be selected according to the profile details displayed to the user through the designated portal 205.

In another exemplary case, the provider (healthcare provider) logs into the system 102 and the provider views a worklist (requests from the users). An appointment is scheduled or is arranged on an on-demand basis through the designated portal for appointment request or the on-demand request. Since the details of the user are stored in the secured database and are confidential, the provider may only see relevant details of the user as required for scheduling the appointment or the on-demand preliminary consultation. The provider however, may request for an access of the details in case of any requirement. Once the request is approved by the system 102 or the user, the provider may access the details.

The consultations may concern the physical health, mental health, education, or therapy of the patient. Vital statistics (details) may be provided to professionals (providers) involved in the consultations.

The provider selects an appointment generated towards the request of the user and reviews the patient's record submitted as part of details. The health care assessment and vital statistics may also be completed by the provider once the appointment is availed by the patient. For example, the provider adds medication or therapy details under the user's details for future reference and record.

The provider additionally, accesses a dashboard over the UI 306, providing an inbox for messages as well as profiles of the users for at least one of the provider handling the task and a subject patient (user). An account is visible as well as a listing of patients and associated patient information. A calendar is available as well as E-Learning modules for the provider and others. Communities and reports are also available for the provider.

The system 102 further comprises a smart module enabling the system 102 to learn during at least telemedicine sessions with the patient/user. The system learns from medical records, including those with IC9/10 and CPT codes, from each consult that takes place. The system learns vital measurements from the electronic devices of the users and providers and may learn non-medical record data from consultations from specific keywords in notes and from consultation forms and documents. Such smart module enables system to suggest further consultations to each of the user and the provider and also to prioritize the appointments.

For example, the system 102 may learn on frequency of specific consultations for the user and frequency of medical records and may further flag potential readmission consultations. The system 102 may learn on types of consultations and frequency to recommend multi-provider/disciplinary consults to all the users including each of the patient and the provider.

The monitoring module of the system 102 learns on types of E-learning Modules that the patient starts and completes according to the learning request. Regarding communities from patients, the system learns about different forum interactions and detects favourites and related activity for providing auto-recommendation to the user. the auto-recommendations may be provided through notifications.

The system 102 also provides designated portals 205 for the benefit of the patient/user as well as the provider. As the provider is assisting the patient, the system 102 may recommend specific consultations based on correlation patterns from previous patients and further based on persistently observed single user consultation patterns. The system 102 may recommend E-Learning Modules and Communities based on the types of consultations, community interaction, and modules started and completed from many users. The system 102 may also recommend measuring of a patient's vital signs on a specific time frame if remote monitoring for the patient has been arranged. Recommendations for obtaining patient vital statistics may be based on vital progressions and measurements of other patients.

Through the designated portals 205 appropriate for the provider, systems 102 and methods (400 and 500) may recommend the physicians schedule follow-ups based on patient data from other users. Percentage of probability may be shown. Physicians may also receive recommendations to schedule multi-provider consults based on patient data from other users, again with percentage of probability shown in some embodiments. The system may also recommend that physicians use specific forms based on other similar consultations.

Physicians (providers) are warned and advised on potential readmission or repeat causes consultations based on correlation pattern of other user medical records. Percentage of probability may be shown over the designated portal. The system 102 may detect a change in an existing patient's vital statistics from remote monitoring and may recommend follow-up based on other users' data. Percentage of probability may be shown.

The system 102 may recognize readmission behaviour based on physician conduct. If the system 102 detect that multiple patients that have consulted with a particular physician for the same complaint during a short period of time, this potentially noteworthy may be documented and reported to the appropriate administrative units.

Similarly, if the system 102 identifies a pattern detected for a physician of sending patients to an emergency room each time a specific type of consultation takes place, the platform will notify the physician of the percentage of probability. Systems 102 and methods (400 and 500) provided herein are directed to managing healthcare costs in addition to improving patient care.

Provider lockout portal (designated portal 205) may warn and advise providers via messaging of upcoming scheduled consultations over the UI of the electronic device. System 102 may consider request from the provider, in case the provider desires to cancel or reschedule 24 hours prior to the consultation.

Through the feedback designated portal, the system 102 may place a strike on a provider's record if the provider misses a scheduled appointment. If three strikes occur, the provider will be locked out. The provider may have an audit to regain access.

Through the feedback designated portal, the system may 102 also detect that a provider may miss a scheduled appointment based on a determination by the system that a patient has waited three to five minutes in a waiting room. The system 102 may then notify the patient/user to look for an alternative on-demand provider to consult with immediately or to make a new appointment with a different provider.

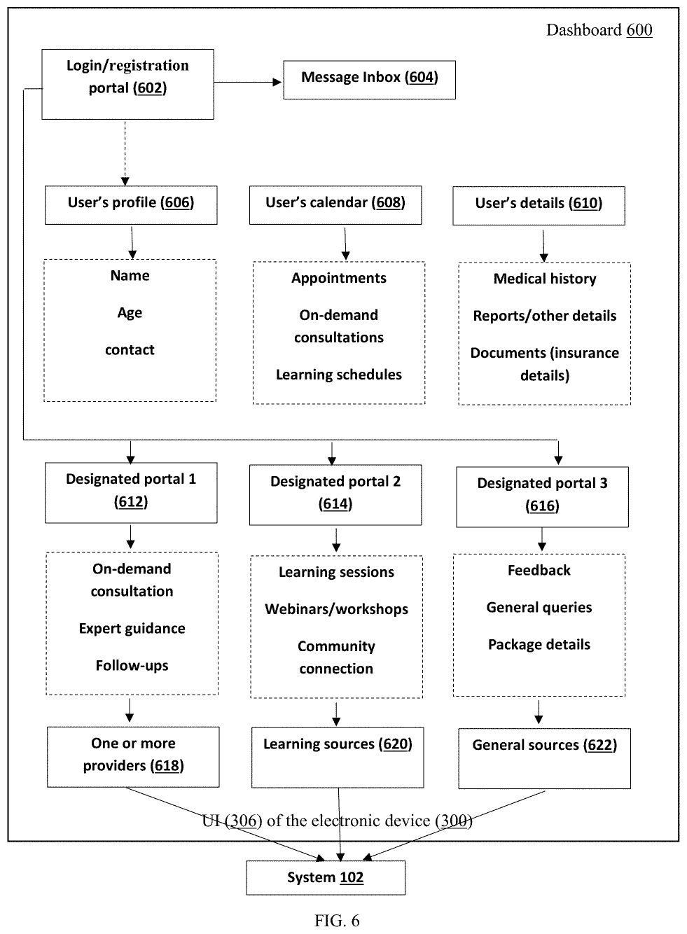

In an exemplary embodiment, FIG. 6 shows a dashboard 600 displayed over the UI 306 of the electronic device 300 of the patient (user). The dashboard 600 is an exemplary dashboard supporting understanding of the invention and may also comprise plurality of non-exhaustive additional features (not displayed on the dashboard 600).

The dashboard 600 comprises login/registration portal 602 for registering the user. The dashboard comprises a message inbox 604 for the user for showing a list of messages towards appointment, reminders, payment details, recommendations and alike.

The dashboard 602 further displays user's profile 606 showing information such as name, age contact and alike. The dashboard also displays a user's calendar 608 showing appointments, on-demand consultations, learning schedules and alike. The dashboard 600 also displays user's details such as medical history, reports/other details, documents (such as insurance documents) and alike.

Through the UI 306, the user may add his personal and contact information as well as gender and date of birth which may not be accessible via the “sign up” screen that may be presented earlier. The UI 604 also allows patients/users and others to change personal information of the patients/users. The patient/user may add a photograph to his/her profile by activating browse.

Through the dashboard, the patient may also check different health categories that the system covers through a static health category menu (not shown). The categories include general health, speech, language and motor development, emotional and mental health, mind, body and nutrition, education, advocacy, and tutoring & coaching. The user may select on an e-learning button to go directly to e-learning resources to learn more.

The dashboard 600 may also support a guest login for guiding first time users. After entering on guest page through the UI 306, the UI 306 may direct the guest (user) to a page that describes the platform and a questionnaire that they would answer to lead them to the correct area of the platform.

The user may also visit an iOS app store and an Android play store to download a mobile version of the system 102. From the guest page the user may navigate to “Sign up”, “Sign in”, “About Us” or “Contact Us” pages. User may subscribe to email updates accessed through message inbox 602. Emails will be sent to them containing information on telemedicine benefits, consult specialties, e-learning lessons, communities and upcoming webinars.

After login, the user may select any of the designated portal (1) 612, designated portal (2) 614 or the designated portal (3) 616. The designated portal (1) connects the user with one or more providers 618 for one of the on-demand consultation, expert guidance, follow-ups and alike. The designated portal (2) connects the user with learning sources 620 for one of the learning sessions, webinars/workshops, community connection and alike. The designated portal (3) connects the user with general sources 622 for one of the feedback, general queries or package details. The designated portals are integrated with the system 102.

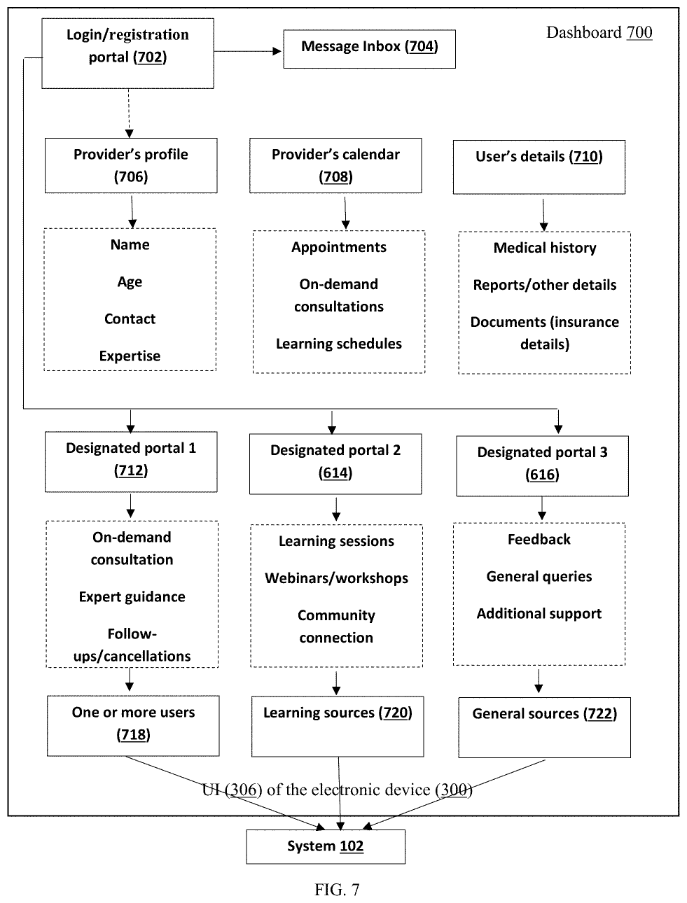

In an exemplary embodiment, FIG. 7 shows a dashboard 700 displayed over the UI 306 of the electronic device 300 of the patient (user). The dashboard 700 is an exemplary dashboard supporting understanding of the invention and may also comprise plurality of non-exhaustive additional features (not displayed on the dashboard 700).

The dashboard 700 comprises login/registration portal 602 for registering the provider. The dashboard comprises a message inbox 704 for the provider for showing a list of messages towards appointment, reminders, patient's communications, recommendations and alike.

The dashboard 700 further displays provider's profile 706 showing information such as name, age contact, medical expertise and alike. The dashboard 700 also displays the provider's calendar 708 showing appointments, on-demand consultations, learning schedules and alike. The dashboard 700 also displays user's details 710 to the provider such as medical history, reports/other details, documents (such as insurance documents) and alike.

After login, the provider may select any of the designated portal (1) 712, designated portal (2) 714 or the designated portal (3) 716. The designated portal (1) connects the provider with one or more users 718 for providing one of the on-demand consultation, expert guidance, follow-ups and alike. The designated portal (2) connects the provider with learning sources 720 for one of the learning sessions, webinars/workshops, community connection and alike. The designated portal (3) connects the provider with general sources 722 for one of the feedback, general queries or any additional support. The designated portals are integrated with the system 102.

Once the provider selects a patient from a dropdown box over the dashboard 700 (not shown), the provider may select the patient and may view for details on the patient's appointments. By default, the dropdown will show patient name which is selected in a Patients Listing Page. In scheduled appointments, the provider may view scheduled appointments for the patient that have been approved by the provider involved. By clicking on Start Now button in scheduled appointments, the provider may enable a consultation with the user on scheduled date and time of the appointment. The Start Now button may be visible five minutes in advance of the appointment time. The provider may also reschedule or cancel the appointment 24 hours prior to the date and time without any penalties.

In requested appointments, the provider may view appointments that have been requested for the particular patient, and that are pending for approval by providers. The provider may check appointment status at this point. If accepted, the appointment will be moved to scheduled appointments. If declined, status will be “declined” and may display options. In the recommended follow-ups section, such follow-up action may be suggested by the provider after previous appointment is completed. If a provider does not recommend a follow-up at the end of an appointment, the system may automatically recommend a follow-up based on behaviour on the system for that particular patient. A recommended follow-up would be to consult a specific specialty provider or to consult a similar provider that fits schedules of patient.

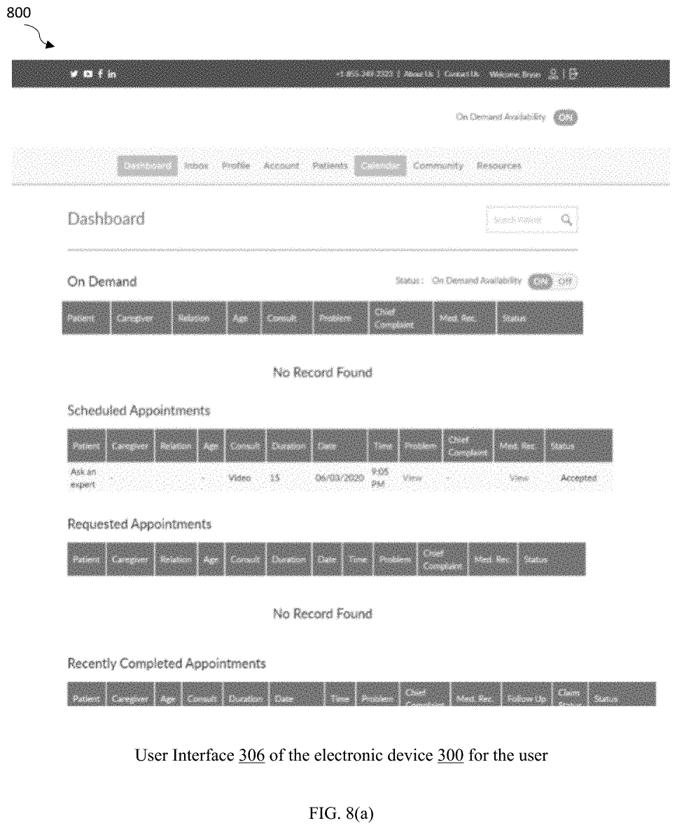

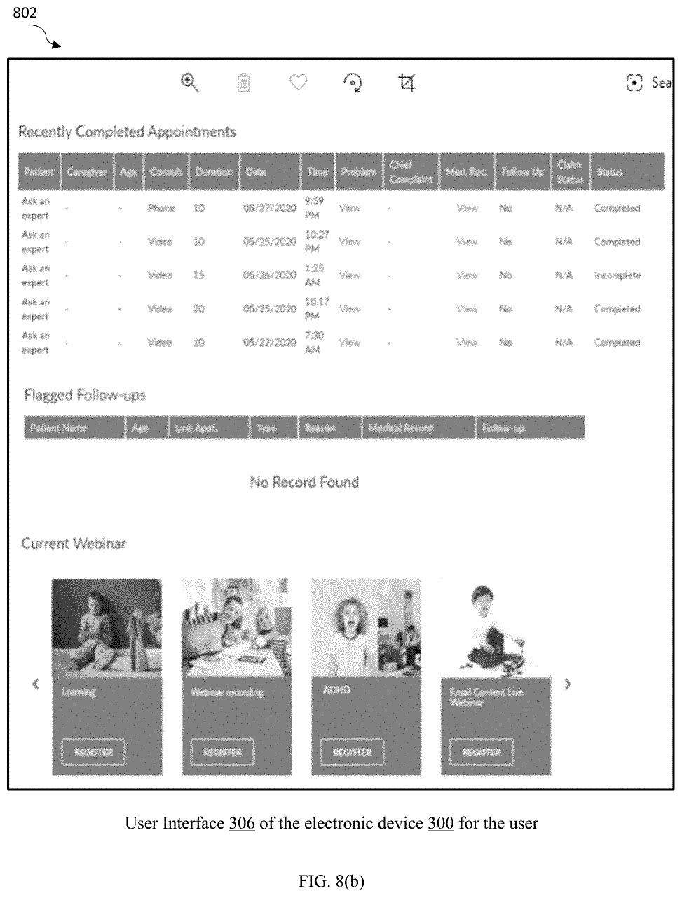

FIG. 8(a) illustrates an exemplary view 800 of the UI 306 of the electronic device 300 facilitating healthcare services to the user, in accordance with an embodiment of the present subject matter.

The UI 306 is providing options to the user such as dashboard, inbox, profile, account, patients, calendar, community and resources. The patient may book the on-demand appointment (preliminary consultation). The UI 306 also shows tabs for scheduled appointments, requested appointments, recently completed appointments.

FIG. 8(b) illustrates another exemplary view 802 of the UI 306 of the electronic device 300 facilitating healthcare services to the user, in accordance with an embodiment of the present subject matter.

The UI 306 further shows details of the recently completed appointments, tab for flagged follow-ups. The UI 306 also shows learning resources for the user such as current webinar.

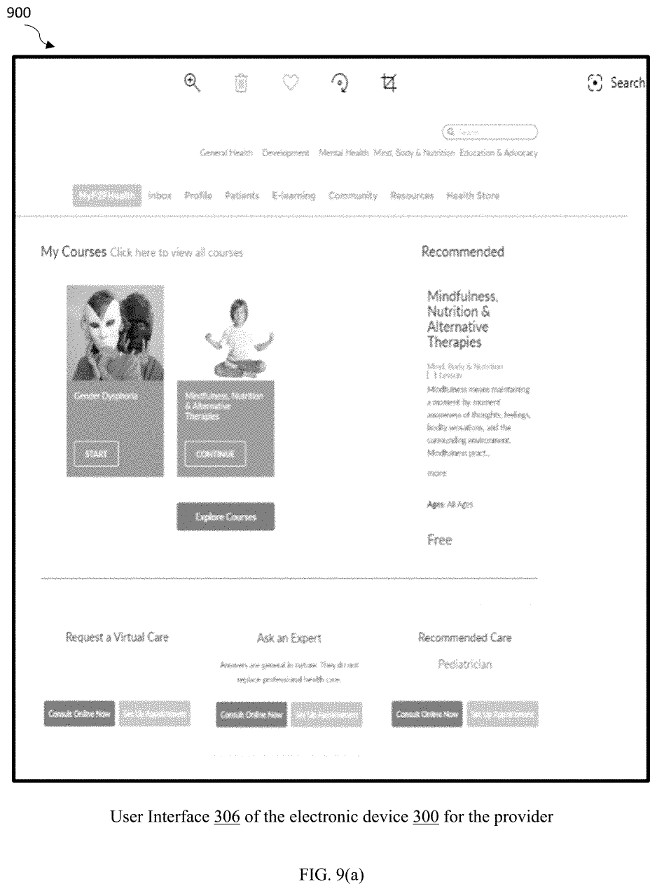

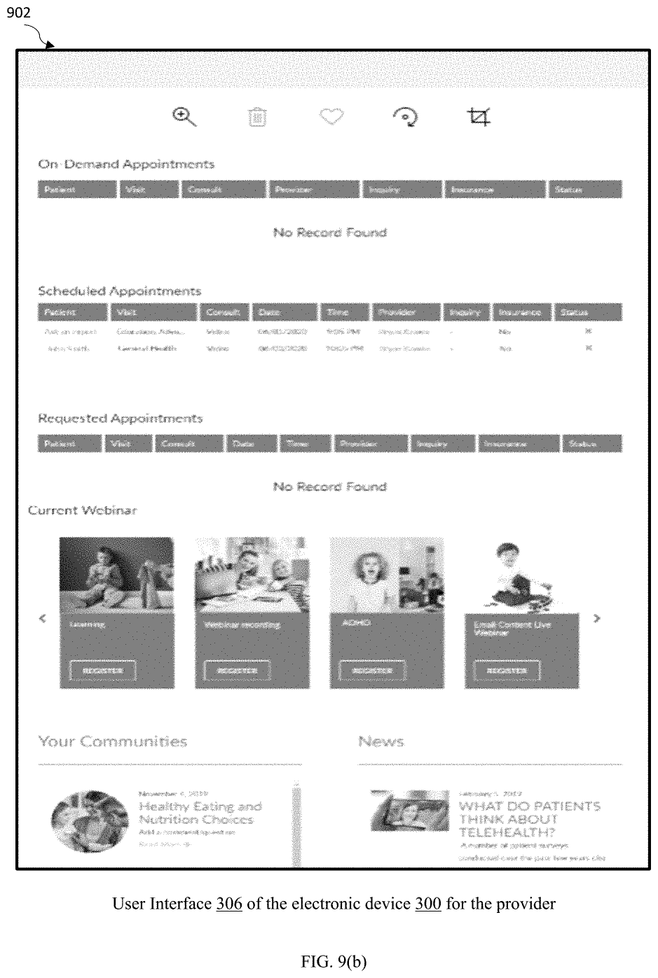

FIG. 9(a) illustrates an exemplary view 900 of the UI 306 of the electronic device 300 facilitating healthcare services through the providers, in accordance with an embodiment of the present subject matter.

The UI 306 is providing options to the user such as inbox, profile, patients, E-learning, community, resources and health store for the provider. The UI 306 also shows tabs for requesting a virtual care, ask an expert, recommended care along with recommended learning options.

FIG. 9(b) illustrates another exemplary view 902 of the UI 300 of the electronic device 300 facilitating healthcare services through the providers, in accordance with an embodiment of the present subject matter.

The UI 306 shows tabs for each of on-demand appointments, scheduled appointments, requested appointments for the provider. UI 306 also shows tabs for current webinar, your communities and news for the providers.

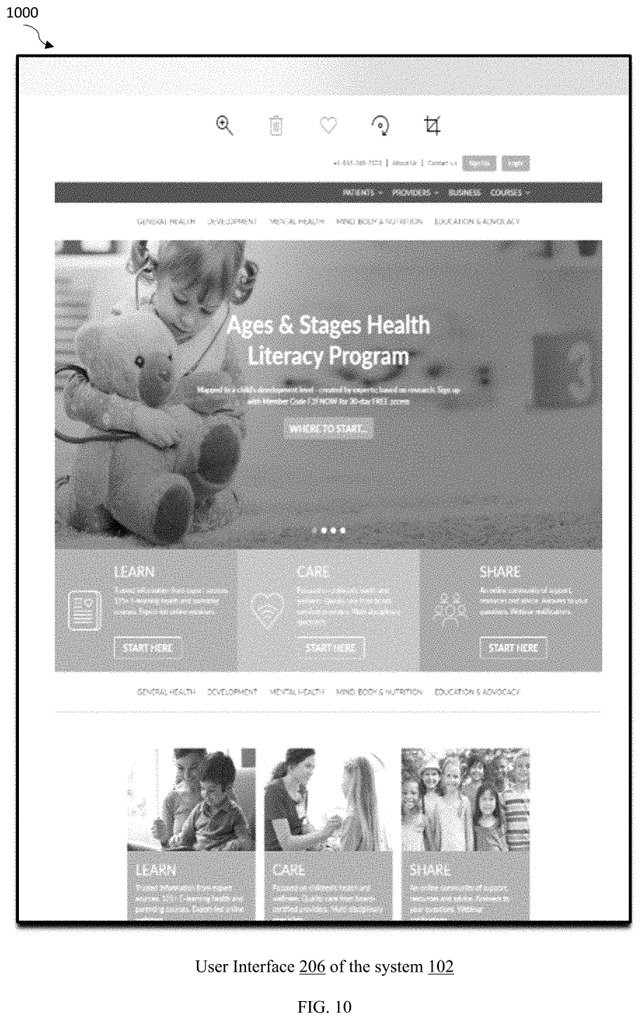

FIG. 10 illustrates an exemplary view 1000 of the UI of the system 102 facilitating healthcare services to the users through the providers, in accordance with an embodiment of the present subject matter.

The UI 206 shows options enabling learn, care and share through the system 102. The UI 102 provides the login for each of the patient and the providers and also provides an option for courses. The UI 102 has the designated portals for each of the general health, development, mental health, mind body and nutrition, and education advocacy.

Each of the views 800, 802, 900, 902 and 1000 are exemplary and are used for a purpose of illustration without limiting the scope of the invention. The working of the system 102, electronic device 300, method 400 and the method 500 will be understood according to details as discussed above.

Exemplary embodiments discussed above may provide certain advantages. Though not required to practice aspects of the disclosure, the advantages may include those provided by the following features.

Some embodiments of the storage system 102 and the method 400 and method 500 may be configured to schedule on-demand preliminary consultations or expert consultations according to the user's preference and requirements without actually requiring any physical visit. The schedule may be generated according to the user's preference and may be accessed in plurality of modes.

Some embodiments of the system 102 and the method 400 and method 500 may auto-generate recommendations, alarms, reminders for the user and the provider in order to facilitate a smart healthcare service process.

Some embodiments of the storage system 102 and the method 400 and the method 500 may monitor details of the user and may flag the user in case of any emergency.

Some embodiments of the storage system 102 and the method 400 and the method 500 may support a learning by offering wide ranges or learning resources to enhance knowledge in an area of the user's interest.

Some embodiments of the storage system 102 and the method 400 and the method 500 may support a communication with other community members for a discussion on common interest areas.

Although implementations for the storage system 102 and the method 400 and the method 500 for facilitating the healthcare services, it is to be understood that the appended claims are not necessarily limited to the specific features or methods described. Rather, the specific features and methods are disclosed as examples of implementations for assigning dynamic operation of devices in a communication network.

1. A system facilitating healthcare services, the system comprising:
a User Interface (UI), configured with a plurality of designated portals, designed for providing the healthcare services;
a memory;
a processor coupled to the memory, wherein the memory stores a plurality of instructions to be executed by the processor, wherein the processor is configured to:
receive, from a user, through the UI, a request for availing a healthcare service through one or more providers, wherein the request comprises at least one of a learning request or a consult request;
direct, the at least one of the learning request and the consult request to a designated portal of the plurality of designated portals selected by the user, wherein the designated portal controls display of the details of the user to the one or more providers, according to the at least one of the learning request and the consult request; and
generate, a request result for at least one of the learning request and the consult request, through the designated portal, wherein the request result is generated in a preselected mode of access and according to a user's preference, such that the user avails the healthcare services by accessing the request result.

2. The system as claimed in claim 1, wherein the user comprises a patient, a guardian of a minor patient, a family member of the patient, wherein the user is a registered user provided with user credentials for accessing the system.

3. The system as claimed in claim 1, wherein the one or more providers comprises at least one of medical experts, non-medical experts, doctors, medical counsellors, non-medical counsellors, psychologists, trainers, and wherein the one or more providers are registered providers accessing the system, through an electronic device, by using login credentials of the providers generated by the system.

4. The system as claimed in claim 1, wherein the request comprises a prepaid package request for availing the healthcare service, wherein the prepaid package comprises at least one of the learning request or a medical assistance request.

5. The system as claimed in claim 1, wherein the consult request comprises at least one of a preliminary medical guidance request, an appointment request for expert medical consultation with one or more providers, reschedule of one of the learning request or the medical assistance request, a cancellation of one of the learning request or the medical assistance request, a follow-up appointment request for expert medical consultation with one or more providers, wherein the learning request comprises a request for accessing a knowledge resource configured within the system, e-learning request, a webinar, or a workshop.

6. The system as claimed in claim 1, wherein the designated portal comprises an on-demand medical guidance portal, a medical expert consultation portal, a health monitoring portal, an e-learning portal, a message portal, a calendar portal, a payment portal, a user profile portal generated after registration of the user for displaying details of the user according to the request, a help portal, a feedback portal, a review portal.

7. The system as claimed in claim 1, wherein the details of the user are stored in a database configured in the system, wherein the details comprises at least one of personal details of the user, a medical history of the user, medical reports or records of the user, insurance details of the user, contact details of the user.

8. The system as claimed in claim 1, wherein the processor is further configured to:
prioritize, the request for a follow-up appointment received as the consult request, according to the details of the user, wherein the details comprises medical history of a patient, frequency of appointment of the patient, health monitoring results as generated by the system; and
schedule, the follow-up appointment according to the prioritizing, wherein the prioritizing sets an order number of the follow-up appointment in a list of appointments.

9. The system as claimed in claim 1, wherein the processor is configured to:
identify, through a monitoring portal of the plurality of designated portals, correlation patterns according to the details of the user and previously generated request results for the user; and
recommend, a subsequent request result according to the correlation pattern, wherein the subsequent request result comprises a follow-up appointment with one or more medical expert or non-medical expert, or learning schedules.

10. The system as claimed in claim 1, wherein the request result comprises at least one of a preliminary consultation for an on-demand medical guidance received as the medical assistance request, a medical expert consultation towards a medical issue, with one or more providers for a medical appointment request received as the consult request, a medical report for a health monitoring request received as the medical assistance request, a list of courses, webinars, workshops, community learning, generated for the request received as the learning request, and wherein the user's preference comprises a preference in selection of a provider from a list of the providers for availing the healthcare services.

11. The system as claimed in claim 1, wherein the preselected mode of access comprises one of an audio mode, a video mode, a text mode, an email mode, wherein the request result comprises one of a real-time request result or a time-slot defined request result.

12. The system as claimed in claim 1, comprising a communication module communicatively coupled to the processor, configured for establishing a communication between the user and the system, wherein the communication module also enables a communication between the user and the one or more providers assisting in availing of the request result.

13. The system as claimed in claim 12, wherein the communication module is configured to trigger each of an alarm towards a medical emergency or a non-medical emergency, and reminders for availing the healthcare service through request results.

14. An electronic device facilitating healthcare services, the electronic device comprising:
a User Interface (UI)
a memory;
a processor coupled to the memory, wherein the memory stores a plurality of instructions to be executed by the processor, wherein the processor is configured to:
register, through the UI, details of the user for submitting a request for availing a healthcare service through one or more providers, wherein the request comprises at least one of a learning request or a consult request;
select, a designated portal of the plurality of portals according to at least one of the learning request and the consult request, wherein the designated portal controls display of the details of the user to the one or more providers, according to the at least one of the learning request and the consult request; and
receive, a request result for at least one of the learning request and the consult request, through the designated portal, wherein the request result is accessed by a user in a preselected mode of access, wherein the request result is received according to a user's preference.

15. The electronic device as claimed in claim 14, wherein the user comprises a patient, a guardian of a minor patient, a family member of the patient, wherein the user is a registered user provided with user credentials for accessing the system.

16. The electronic device as claimed in claim 14, wherein the one or more providers comprises at least one of medical experts, non-medical experts, doctors, medical counsellors, non-medical counsellors, psychologists, trainers, and wherein the one or more providers are registered providers accessing the system, through an electronic device, by using login credentials of the providers generated by the system.

17. The electronic device as claimed in claim 14, wherein the consult request comprises at least one of a preliminary medical guidance request, an appointment request for expert medical consultation with one or more providers, reschedule of one of the learning request or the medical assistance request, a cancellation of one of the learning request or the medical assistance request, a follow-up appointment request for expert medical consultation with one or more providers, wherein the learning request comprises a request for accessing a knowledge resource configured within the system, e-learning request, a webinar, or a workshop.

18. The electronic device as claimed in claim 14, wherein the designated portal comprises an on-demand medical guidance portal, a medical expert consultation portal, a health monitoring portal, an e-learning portal, a message portal, a calendar portal, a payment portal, a user profile portal generated after registration of the user for displaying details of the user according to the request, a help portal.

19. The electronic device as claimed in claim 14, wherein the details comprises at least one of personal details of the user, a medical history of the user, medical reports or records of the user, insurance details of the user, contact details of the user.

20. The electronic device as claimed in claim 14, wherein the request result comprises at least one of a preliminary consultation for an on-demand medical guidance received as the medical assistance request, a medical expert consultation towards a medical issue, with one or more providers for a medical appointment request received as the medical assistance request, a medical report for a health monitoring request received as the medical assistance request, a list of courses, webinars, workshops, community learning, generated for the request received as the learning request, wherein the user's preference comprises a preference in selection of a provider form a list of the providers.

21. The electronic device as claimed in claim 14, wherein the preselected mode of access comprises one of an audio mode, a video mode, a text mode, an email mode, wherein the request result comprises one of a real-time request result or a time-slot defined request result.

22. The electronic device as claimed in claim 14, comprising a communication module communicatively coupled to the processor, configured for establishing a communication between the user and the system, wherein the communication module also enables a communication between the user and the one or more providers assisting in availing of the request result.

23. An electronic device facilitating healthcare services, the electronic device comprising:
a User Interface (UI) with a dashboard showing a plurality of designated portals;
a memory;
a processor coupled to the memory, wherein the memory stores a plurality of instructions to be executed by the processor, wherein the processor is configured to:
receive, through the UI, a request for availing a healthcare service from one or more users, wherein the request comprises at least one of a learning request or a consult request, wherein the healthcare services are availed by connecting with one or more providers;
select, a designated portal of the plurality of portals according to at least one of the learning request and the consult request, wherein the designated portal controls display of the details of the user to the one or more providers, according to the at least one of the learning request and the consult request; and
generate, a request result for at least one of the learning request and the consult request, through the designated portal, wherein the request result is generated in a preselected mode of access, wherein the request result is generated according to a user's preference. such that the user accesses the request result for availing the healthcare services.

24. The electronic device as claimed in claim 23, wherein the user comprises a patient, a guardian of a minor patient, a family member of the patient, wherein the user is a registered user provided with user credentials for accessing the system.

25. The electronic device as claimed in claim 23, wherein the one or more providers comprises at least one of medical experts, non-medical experts, doctors, medical counsellors, non-medical counsellors, psychologists, trainers, and wherein the one or more providers are registered providers accessing the system, through an electronic device, by using login credentials of the providers generated by the system.

26. The electronic device as claimed in claim 23, wherein the consult request comprises at least one of a preliminary medical guidance request, an appointment request for expert medical consultation with one or more providers, reschedule of one of the learning request or the medical assistance request, a cancellation of one of the learning request or the medical assistance request, a follow-up appointment request for expert medical consultation with one or more providers, wherein the learning request comprises a request for accessing a knowledge resource configured within the system, e-learning request, a webinar, or a workshop.

27. The electronic device as claimed in claim 23, wherein the designated portal comprises an on-demand medical guidance portal, a medical expert consultation portal, a health monitoring portal, an e-learning portal, a message portal, a calendar portal, a payment portal, a user profile portal generated after registration of the user for displaying details of the user according to the request, a help portal, a feedback portal, a review portal.

28. The electronic device as claimed in claim 23, wherein the details comprises at least one of personal details of the user, a medical history of the user, medical reports or records of the user, insurance details of the user, contact details of the user.

29. The electronic device as claimed in claim 23, wherein the request result comprises at least one of a preliminary consultation for an on-demand medical guidance received as the medical assistance request, a medical expert consultation towards a medical issue, with one or more providers for a medical appointment request received as the medical assistance request, a medical report for a health monitoring request received as the medical assistance request, a list of courses, webinars, workshops, community learning, generated for the request received as the learning request, wherein the user's preference comprises a preference in selection of a provider from a list of providers.

30. The electronic device as claimed in claim 23, wherein the preselected mode of access comprises one of an audio mode, a video mode, a text mode, an email mode, wherein the request result comprises one of a real-time request result or a time-slot defined request result.

31. The electronic device as claimed in claim 23, comprising a communication module communicatively coupled to the processor, configured for establishing a communication between the user and the system, wherein the communication module also enables a communication between the user and the one or more providers assisting in availing of the request result.

32. A method facilitating healthcare services, the method comprising:
receiving, from a user, through the UI, a request for availing a healthcare service through one or more providers, wherein the request comprises at least one of a learning request or a consult request;
directing, the at least one of the learning request and the consult request to a designated portal of a plurality of designated portals selected by the user, wherein the designated portal controls display of the details of the user to the one or more providers, according to the at least one of the learning request and the consult request; and
generating, a request result for at least one of the learning request and the consult request, through the designated portal, wherein the request result is generated in a preselected mode of access, wherein the request result is generated according to a user's preference. such that the user avails the healthcare services by accessing the request result.

33. A method facilitating healthcare services, the method comprising:
registering, through the UI, details of the user for submitting a request for availing a healthcare service through one or more providers, wherein the request comprises at least one of a learning request or a consult request;
selecting, a designated portal of the plurality of portals according to at least one of the learning request and the consult request, wherein the designated portal controls display of the details of the user to the one or more providers, according to the at least one of the learning request and the consult request; and
receiving, a request result for at least one of the learning request and the consult request, through the designated portal, wherein the request result is accessed by a user in a preselected mode of access, wherein the request result is received according to a user's preference.

34. A method facilitating healthcare services, the method comprising:
receiving, through the UI, a request for availing a healthcare service from one or more users, wherein the request comprises at least one of a learning request or a consult request, wherein the healthcare services are facilitated through the one or more providers;
selecting, a designated portal of the plurality of portals according to at least one of the learning request and the consult request, wherein the designated portal controls display of the details of the user to the one or more providers, according to the at least one of the learning request and the consult request; and
generating, a request result for at least one of the learning request and the consult request, through the designated portal, wherein the request result is generated in a preselected mode of access, wherein the request result is generated according to a user's preference, such that the user accesses the request result for availing the healthcare services.
